(19) United States
(12) Patent Application Publication
(10) Pub. No.: US 2021/0045690 A1
(43) Pub. Date: Feb. 18, 2021

(54) NEURAL PROBE FOR ELECTROSTIMULATION OR RECORDING AND FABRICATION PROCESS FOR SUCH A PROBE

(71) Applicant: Universitat Basel Vizerektorat Forschung, Basel (CH)

(72) Inventors: Bekim OSMANI, Basel (CH); Tino TOPPER, Freiburg (DE); Bert MUELLER, Embrach (CH); Per Magnus KRISTIANSEN, Mannedoft (CH); Helmut SCHIFT, Biel/Bienne (CH)

(73) Assignee: Universitat Basel Vizerektorat Forschung, Basel (CH)

(21) Appl. No.: 16/976,917

(22) PCT Filed: Mar. 1, 2019
(86) PCT No.: PCT/EP2019/055126

§ 371 Date: Aug. 31, 2020

(30) Foreign Application Priority Data
Mar. 1, 2018 (EP) 18159592.7

(51) Int. Cl. A61B 5/00; H01B 1/12; A61B 5/04; A61N 1/05; A61N 1/375

(52) U.S. Cl. CPC A61B 5/6868; H01B 1/124; A61B 5/04001; A61N 1/0534; B82Y 15/00; A61B 2562/028; A61B 2562/0285; A61B 2562/125; A61B 2562/222; A61N 1/37514

(57) ABSTRACT

For improving the electroactivity and long-term stability of a neural interface, a novel neural probe (1) is proposed that is formed from a fiber (6), preferably by thermal imprinting, and wherein a polymer thin film (5) is employed for carrying a conducting thin film to be used as a recording or stimulation electrode (4). Due to this specific choice of materials and design, the electrode (4) is rendered compliant with respect to the fiber (6) on a nanometer to micrometer scale and offers a surface that is tailor-made for adhering to nervous tissue.

### TECHNICAL FIELD

The invention concerns a neural probe comprising a carrier body forming a contact area, in particular for contacting nervous tissue such as brain tissue, and at least one electrode arranged within the contact area. The invention further proposes a new approach for achieving a high number of such electrodes.

The invention further relates to a process for fabricating a neural probe featuring a polymer thin film which carries at least one electrode of the probe. The polymer thin film is carried by a carrier body. Preferably, the carrier body of such a probe may form a contact area within which the at least one electrode is arranged.

### BACKGROUND

Neural probes are commonly used to electrostimulate nervous tissue, in particular brain tissue, or to record nerve signals from such tissue. Due to the disruptive progress in the field and the prospects of healing illnesses so far regarded as incurable, the interest of the scientific and medical community in neural probes in particular for stimulating the human brain has been rising constantly in recent years.

In particular for stimulation of deep brain regions, state-of-the-art neural probes offer only a limited number of electrodes, typically not more than eight. As a consequence, it is so far not possible to accurately steer the electrical currents used for stimulating the brain region around the distal end portion of the probe.

Moreover, other state-of-the-art neural probes with a large number of electrodes are fabricated from stiff materials such as silicon, titanium, platinum or iridium. As a result the brain tissue around the probe is damaged over time, as the brain is constantly moving in the skull and hence relatively to the probe. As a secondary result, a dense scar tissue is forming around the probe only several days or weeks after the implantation.

On the other hand, when using soft substrate materials such as silicones (e.g. polydimethylsiloxane), it can be very challenging to insert the probes deeply into the brain, as required for deep brain stimulation probes.

Another un-resolved problem lies in the non-compliance of the electrode surfaces themselves. Very often, the initially high electroactivity of the neural interfaces building up between the electrodes of a neural probe and the brain tissue is observed to degrade rapidly after the probe has been inserted into the brain. Achieving long-term stability of the recording of nerve signals or the electrostimulation in particular of deep brain regions is therefore a challenge yet to be solved.

### SUMMARY

It is thus a first object of the present invention to provide a neural probe that can be introduced deeply into brain tissue and which offers a high number of stimulation or recording electrodes. It is a further object of the present invention to increase the electroactivity and long-term stability of the neural interfaces formed between brain tissue and the electrodes of the probe.

In accordance with the present invention, a neural probe is provided which solves the afore-mentioned problems. In particular the invention proposes a neural probe as introduced at the beginning, which, in addition, is characterized in that an intermediate soft thin film carries the at least one electrode, wherein the soft thin film is carried by a carrier body. In particular wherein the soft thin film may be softer than the carrier body.

For example, the electrode may be patterned directly on and/or within the soft thin film, which is preferably a polymer thin film, e.g. an elastomer. Preferably, however, the electrode may be firmly linked to the soft thin film by an intermediate adhesion promotion layer. The adhesion promotion layer preferably has a sub-micrometer thickness, most preferably below 100 nm, and is preferably formed from a functionalized siloxane based polymer, for example polydimethylsiloxane (PDMS) with thiol functionalized end groups. In cases, where the electrode is formed within the soft thin film, an intermediate adhesion promotion layer is not necessarily required.

Additionally, insulation layers may be used to electrically insulate parts of the material forming the electrode; thus precise neural interfaces may be defined.

As a major benefit of the present invention, the soft thin film acts as an intermediate buffering layer between the relatively stiff carrier body of the probe and the electrode. The soft thin film may thus compensate mechanical loads acting on the electrode and minimize physical and mechanical mismatches between the neural tissue and the electrode. As a result, the electrode is rendered compliant and/or deformable on a nanometer to micrometer scale. This mechanical functionality is highly beneficial for achieving a long-term stable electroactivity of the neural interface that forms between the electrode and the brain tissue. As a result, the electrode remains in close contact with the tissue, even when the tissue is moving or deformed. According to the invention, a thickness of the soft thin film of less than 5 μm may be sufficient for providing such functionality. In particular cases, the soft thin film may show a thickness in the submicrometer range.

The contact area of the support body holding the electrodes can be located at a distal end of the probe or at side surfaces of the probe; it may be planar or convex or concave. This approach offers a large freedom of design for spatially arranging the electrodes.

In addition, a probe according to the present disclosure has the benefit of a potentially large surface area for electrostimulation and/or recording of nerve signals. The underlying reason is that the soft thin film may show a surface corrugation, as will be explained in greater detail below, and this corrugation can increase the effective surface area of the electrode to be brought into contact with brain tissue. A corrugation of the electrode has been found to be also beneficial for increased electroactivity of the neural interface between the electrode and the brain tissue; in particular the neural cell attachment on the electrode is promoted and hence the signal transmission is enhanced.

According to the invention, there exist numerous further advantageous embodiments solving the aforementioned problems, which are outlined in the sub-claims and will be described in detail in the following.

For example, one embodiment suggest to use the neural probe described herein as an electrostimulation or recording probe. Hence in this embodiment, the at least one electrode is either an electrical stimulation or an electrical recording electrode. In some applications, the electrode can also be used for both electrostimulation and recording.

A general challenge is to conceive an efficient way of fabricating neural probes offering a high temporal and spatial signal resolution at reasonable costs. For this purpose, one preferred embodiment suggests that the carrier body of the probe is a fiber; a typical outer diameter of the fiber may be 300 μm.

In fact, it is preferably if an outer diameter of the fiber is less than 0.8 mm and/or more than 0.05 mm. Such sizes have been found to be ideal for achieving a robustness that is sufficient for handling and insertion of the fiber into the brain and which minimizes the damage to the brain resulting from the insertion.

In particular when employing several of said neural probes in a bundle, as will be explained later, it is most preferably if the outer diameter of the fiber is less than 0.5 mm and more than 0.05 mm.

For best compliance of the electrodes it is further proposed that the soft thin film is elastic. This ensures that the electrode surface can follow tiny movements of the brain tissue. In addition, the thinness of the fiber will enable the electrodes of the probe to follow tiny pulsatile movements of the brain tissue, which are in the range of some hundred micrometers. The soft electrode surface delivered by the invention thus mimics the mechanical properties of the neural tissue.

Additionally or alternatively it is of great benefit if the soft thin film has a Young's modulus, which is at least 10³ times smaller, preferably 10⁴ times smaller, than a Young's modulus of the carrier body of the probe. With such a feature, excellent compliance of the electrode can be achieved. For example, typical material for the carrier body of the probe may show a Young's modulus in the order of 5 GPa, whereas, according to the invention, the material of the soft thin film may show a Young's modulus of less than 5 MPa, preferably less than 2 MPa, preferably less than 1 MPa, for example less than 500 kPa.

According to another embodiment, the at least one electrode may be advantageously formed as a thin film electrode, preferably of a metal or metal alloy. To enable the desired compliance and conductivity, the thickness of the thin film electrode is preferably less than 50 nm, most preferably less than 20 nm.

The at least one electrode may be linked to the soft thin film by an intermediate adhesion promotion layer, in particular consisting of thiol-functionalized PDMS. For example, when using gold as the electrode material, a suitable adhesion promotion layer can be a thin film of thiol functionalized PDMS with a thickness of less than 100 nm, and preferably more than 10 nm. In particular, the electrode may be embedded/incorporated into the adhesion promotion layer.

Using this approach, excellent electrostimulation with minimum size of the probe can be achieved at low fabrication costs. Moreover, with this approach standard micro-patterning techniques for defining a large number of features and/or electrodes on the probe can be employed.

For improving the electroactivity of the neural interface, another preferred embodiment suggests that the at least one electrode forms a corrugation. This corrugation may be in the microscale, but is preferably in the nanoscale. Alternatively or additionally, the electrode may also follow a micro- and/or nanoscale corrugation; the corrugation may thus be the result from a corrugated structure of the probe, for example the soft thin film.

In summary (as will be explained in more details below), the corrugation of the electrode can be the result of, for example, (i) a corrugation formed in the carrier body of the probe (e.g. by thermal imprinting/hot embossing, UV-assisted imprinting or other suitable molding techniques—see below); (ii) a corrugation formed in the soft thin film (e.g. by treating the film with a plasma or UV-radiation); or (iii) it may be formed during deposition of the electrode layer. The latter alternative may be achieved, for example, by forming/depositing the at least one electrode as a (in particular sputter-deposited) thin film with intrinsic, preferably compressive, stress.

The corrugation described above may show a periodicity between 0.1 μm to several μm and/or an amplitude (depth of the corrugation) in the order of a few nanometers to several micrometers.

In particular, the corrugation just described may be in the form of a surface corrugation of the outer surface of the electrode to be brought into contact with brain tissue.

A surface corrugation of the metal electrode may also increase the stiffness of the electrode in particular directions while increasing it in other directions. This property is beneficial for designing the mechanical compliance of the electrode spatially.

The corrugation may show nanoscale ripples or wrinkles. In this case, it is preferable if the nanoscale corrugation and/or the nanoscale ripples run along a major direction which forms an angle to a longitudinal axis of the fiber/the probe. By such an arrangement, the electrode can be made comparably stiff in the transversal direction of the fiber and highly compliant/deformable in the longitudinal direction of the fiber/the probe (i.e. in a direction, in which bending of the fiber is most pronounced).

The corrugation or structure, in particular said ripples, may be formed on elevated as well as lower parts of a microscale structure (to be detailed later) in the contact area, for example on mesas or in bottoms of trenches between elevated structures.

In a highly preferred embodiment, the soft thin film itself features a, preferably nanoscale, corrugation and this corrugation is covered by the at least one electrode. In such an embodiment, it is preferable when the at least one electrode is a thin film electrode of homogenous thickness. In this case, the thin film electrode may repeat the corrugation of the soft thin film on its outer surface.

For example when sputtering the electrode as a thin film of homogenous thickness directly onto a corrugated film acting as the soft thin film of the probe, the stress in the sputtered layer can be adjusted in such a manner, that the sputtered thin film follows the corrugation of the soft thin film. Hence, the surface area of the sputtered film to be used as an electrostimulation and/or recording electrode may be greatly increased, even when employing only a nanoscale corrugation.

Yet another embodiment increases the versatility of the possible electrode arrangements by proposing that the contact area forms a microscale structure. In other words, the micro-structure may be formed in the carrier body of the probe, in particular in a fiber. The microscale structure may be planar or convex or concave; it may be formed in the surface of the contact area, for example as a protrusion or a recess; the microscale structure may also show various shapes, in particular circular or rectangular ones. While the nanoscale structures of the probe may be preferably soft and flexible, the microscale structures may be preferably stiff and/or rigid.

One functionality achievable with such a micro-structure is to provide an enlargement of the contact area itself; another functionality of the micro-structure is to control the formation and orientation of the corrugations described before (see below).

When forming such a microscale structure in the contact area of the probe, it is further preferable when the at least one electrode and/or the soft thin film is/are deposited on the microscale structure. Alternatively, the at least one electrode and/or the soft thin film may be arranged on the microscale structure, for example during assembly of the probe. In particular, the assembly or deposition may be such that the microscale structure is covered by the soft thin film.

Another aspect of the invention is a new approach for achieving anisotropic compliance of the electrodes. For this purpose, the invention suggests to employ corrugations on a micrometer to nanometer scale. For example, using the processes discussed herein, it is possible to form nanoscale corrugations with periodicities ranging from 400 nm to 3 μm and amplitudes (peak-to-valley) in the order of 10 nm to 200 nm.

One highly efficient way of forming such corrugations is to deposit the soft thin film on deliberately designed micro-structures. Following this approach, one embodiment suggests that the microscale structure in the contact area of the probe features a dimension in a first direction that is larger than a dimension in a second direction. Preferably the dimension in the second dimension may be less than 100 μm. By such designs, it can be achieved in particular that the nanoscale ripples run approximately parallel to the second direction. Hence, the orientation of the corrugation (and the resulting compliance) may be accurately designed.

According to the invention, the contact area of the probe may be formed by thermal imprinting (also referred to as hot-embossing), UV-assisted imprinting or other suitable molding techniques. When using thermal imprinting, thermoplastic materials can be used (which may be the material of the carrier body of the probe itself), whereas for UV-assisted imprinting, suitable materials are those which can be crosslinked by exposure to UV-wavelengths (and which may be applied to the carrier body of the probe by an additive process). In both cases micro- as well as nanoscale structures may be defined within the contact area.

For thermally imprinting the contact area, flat stamps may be used, applied by flat stamp imprinting, or curved stamps, in particular applied by roller imprinting. For example, the contact area (and possibly a micro- and/or nano-structure contained in that area) may be formed by thermal imprinting with a heated stamping-tool (pressed into the carrier body) or by so called roll-embossing, in which a heated (and curved) stamping-tool is rolled over the carrier body; such processes are particularly useful, when a fiber is used as the carrier body of the probe.

In particular when employing a fiber as the carrier body of the probe, the contact area may thus be formed from the fiber material directly. In both cases, the formation can be such that a surface area of the fiber is locally increased at a location of the contact area. Hence a larger area can be used for arranging the electrodes of the probe. To avoid damage to the delicate tissue of the brain, the distal fiber ends may be rounded, for example likewise by thermal imprinting.

In yet another beneficial embodiment two contact areas are formed on opposing side areas of the fiber. Each of these two contact areas may feature at least one electrode. By this approach the versatility of the neural probe is greatly enhanced; in particular it is possible to generate and steer an electrical field which builds up between the two electrodes located on different sides of the probe.

The fiber used as the carrier body of the probe may have a core which is electrically conducting. Alternatively or additionally, the fiber, in particular in the core or a cladding, may also feature an electrical wiring. By these measures, electrical signals can be safely guided from recording electrodes at the distal end of the fiber to its proximal end.

In another embodiment, which may also be combined with the one just described before, the core is stiffer than a surrounding cladding of the fiber. Thus the stiff core can provide rigidity to the probe for penetrating the brain tissue, while a soft cladding of the fiber can minimize the damage to the tissue. Such a stiff core may be formed from a stiff polymer or metal wire(s), for instance.

Another highly advantageous embodiment suggests that the core is additionally or alternatively extractably arranged within the cladding. The resulting probe can thus be introduced into the brain with the extractable core introduced into the cladding (providing a high stiffness) and the core may be extracted afterwards to render to prober more flexible and soft. In such an embodiment, an electrical wiring, for example embedded in the fiber cladding, may be used for providing the necessary signal transmission from the electrode at the distal end of the probe to the proximal end of the probe.

The fiber, from which the probe can be fabricated efficiently, is preferably a polymer fiber.

The soft thin film can be a polymer thin film. In particular the soft thin film may be an elastomer thin film. Generally, it is preferable if the Young's Modulus of the soft thin film, in particular the soft polymer thin film, is at least a factor of 100, most preferably a factor of 10³, lower than the Young's Modulus of the carrier body of the probe, in particular lower than a Young's Modulus of the polymer of the fiber.

The neural probe according to the invention can be further improved by using a siloxane as the material for the soft thin film. Preferably the soft thin film is made from a polydimethylsiloxane (PDMS), as this material is biocompatible, almost incompressible, and offers an almost ideal elastic behavior. The material for the soft thin film may contain free thiol-groups to create covalent bonds, e.g. thiol-functionalized PDMS. For instance, covalent bonds may be formed between the soft thin film and metal atoms and clusters embedded within the soft thin film.

The polymer of the fiber may be an organic polymer, preferably polyurethane (PU) or polyethylene terephthalate (PET). These materials are likewise biocompatible and can be conveniently formed by thermal imprinting.

In many applications, it may be of great advantage if the contact area of the probe features an array of electrodes. In such an array, each of the electrodes may be arranged on a microscale mesa. By such a feature, safe contact of the electrode and the tissue can be safeguarded. In such a situation, it is most preferably when neighboring electrodes are separated by a microscale trench. This allows a clear distinction of signals recorded by individual electrodes.

Another advantage of using an array of electrodes is the possibility of precise steering of electrical stimulation currents or fields, respectively. For example using the approach proposed herein, it is possible to control different groups of electrodes within the array. Moreover, when using multiple probes (for example configured to form a bundle, as explained below) electrodes on neighboring probes may be used in concerted fashion.

In one particular advantageous embodiment, on each of two sides of the probe an array of electrodes is located, with each of the arrays featuring at least two electrodes, which can be stimulation and/or recording electrodes. In this situation, it is preferable if each of the arrays can be independently controlled for accurately steering the stimulation fields on each side of the probe independently.

In a neural probe according to the invention, the soft thin film may have a thickness of less than 10 um, preferably of less than 2 um. Such a small thickness has been found to be sufficient for ensuring the compliance of the electrode.

The at least one electrode of the probe may have a thickness of less than 50 nm, preferably of less than 20 nm, most preferably of less than 10 nm. In particular in the latter case, the at least one electrode may be patterned not as a uniform film but as a network of connected islands/clusters on the soft thin film. In a preferred embodiment, the electrode consists of electrically connected islands of gold employed to an adhesion promoting film, for example thiol-functionalized PDMS with a thickness of less than 100 nm, and most preferably of more than 10 nm. Such an electrode can be embedded in the thiol-PDMS adhesion layer such that a highly compliant Au/SH-PDMS matrix is formed.

A network as discussed above can be considered as a mixture between a dielectric and a metallic component. The conductivity o and the dielectric constant ϵ of the mixture show a critical behavior if the fraction of the metallic/conducting component reaches the percolation threshold. This will typically be the case if the thickness of the network reaches a critical thickness. The behavior of the conductivity near this percolation threshold will show a smooth change-over from the low conductivity of the dielectric component to the high conductivity of the metallic component, whereas the dielectric constant will diverge if the threshold is approached from either side.

When the electrode network is undergoing elastic deformations during use of the probe, it could be possibly stretched below the percolation threshold, in which case, the electrode would become non-conductive. Hence, in case the electrode is patterned as a network as described above, it is preferable if the electrode has a thickness above the percolation threshold (i.e. above the critical thickness). By such a dimensioning of the electrode, efficient stimulation and/or recording can be guaranteed while at the same time, the electrode can follow the elastic deformations of the underlying soft thin film without being destroyed or becoming non-conductive. Hence, a convenient long-term stability of the probe, in particular for clinical use, can be achieved.

In order to maintain a high flexibility of the electrode, it is preferable that the electrode has a thickness below four times the percolation threshold (four times below the critical thickness).

For delivering currents to the distal end of the probe or for transmitting electrical signals from that location to the proximal end of the probe, an electrical wiring may be arranged on an outer surface of the fiber; this wiring may be preferably applied by printing or sputtering. Further, the carrier body, in particular the fiber, may feature electrical contacts pads on an outer surface, which are connected to said electrical wiring.

The fiber/the carrier body may also feature an encapsulation which insulates said wiring. In particular, an encapsulation, for example in the form of an electrically insulating layer, may be formed on an outer surface of the carrier body/the fiber such that the wiring is insulated.

The invention discloses also a new approach for delivering a high number of electrodes into a targeted region, in particular a deep brain region. For this purpose, it is suggested to form a neural probe bundle. This bundle may comprise several neural probes as described before or as set forth in the claims, for example at least two of such probes. Such a bundle may thus offer multiple electrodes, each positioned at different axial and/or radial positions along the bundle (and preferably on various of the bundle's probes) and thus provide a high spatial resolution for stimulation and/or recording.

As a major advantage of this approach, a simple fabrication process for the single probes can be repeated to form a highly versatile biomedical device for electrostimulation and recording of nerve signals. For example, the single probes of the bundle may be moved relative to each other, thus allowing manifold electrode arrangements. The single probes of the bundle or the bundle as a whole may be inserted into brain tissue using an insertion tool, which can have an inner diameter of less than 2.0 mm for example.

State-of-the-art neural probes, for example from Boston Scientific, feature only 8 individual electrodes on a probe with an outer diameter of 1.5 mm. Using a bundle of neural probes as suggested here, preferably with much smaller diameters of less than 0.8 mm, most preferably less than 0.5 mm, for example 50 μm, a large number of electrodes can be created, resulting, for example, in up to 1024 different electrical channels (when using an insertion tool with an inner diameter of 2.0 mm).

According to another advantageous embodiment, the carrier body may carry a conductor. By the conductor the electrode can be electrically contacted. In particular, the conductor maybe formed as a conductive track or layer e.g. made of gold and/or platinum and/or titanium. In order to form an adhesive conductor to the polymer fiber/probe it is preferred to form the conductive track by using high-power impulse magnetron sputtering.

The electrode may be formed on and/or within the soft thin film. For instance, the electrode may be formed by metal atoms or clusters, e.g. gold and/or and/or platinum and/or titanium, which are embedded in the soft thin film (embedded electrode). In this case, the electrode may be electrically supplied by the conductor, which has been mentioned before. The conductor may be applied beneath the soft thin film.

The metal atoms or clusters may be covalently bonded to thiol sites within the soft thin film to increase the stability of the electrode.

The carrier body and/or the contact area may be stretchable without tearing apart. In particular enabling a strain of at least 10%, preferably at least 20%, preferably at least 30%, preferably at least 40%, preferably at least 50%, preferably at least 60%, preferably at least 70%, preferably at least 80%, preferably at least 90%, preferably at least 100%, more preferably of more than 100%. In particular, a strain of up to 160% is achievable. In combination with the embedded electrode, the electrode may also be stretched, wherein a conductivity of the embedded electrode is retained or impaired in a tolerable measure.

According to another advantageous embodiment the carrier body may have a micro- and/or nanoscale corrugation, which defines the corrugation of the electrode and/or the soft thin film. The corrugation of the carrier body can be achieved by thermal imprinting and/or UV-assisted imprinting.

The corrugation of the carrier body can be covered with the soft thin film having a constant or almost constant thickness. Alternatively, the carrier body can be covered with a soft thin film having a variable thickness. In cases of variable thickness, the gauges of the carrier body's corrugation can be completely filled out by the soft thin film. By using different heights and thicknesses of the soft thin film it is feasible to alter the nanoscale structure/corrugation of the soft thin film, since thereby the alignment of the wrinkles can be affected.

In the case of a bundle of neural probes as described before, it may be preferable that the single probes have electrical wirings which are insulated by an additional cladding (external, insulated electrical wiring) or which are buried inside the fiber (internal electrical wiring). This avoids short-circuits between neighboring fibers of the bundle.

In accordance with the present invention, there is also provided a fabrication process, which solves the afore-mentioned problem. This process is highly suitable for fabricating a neural probe according to the invention as described before or as set forth in the claims.

In particular there is provided a process as introduced at the beginning, further characterized in that the polymer thin film of the probe is deposited from a gas or liquid phase and cross-linked during deposition. This process-step, which may be referred to as in-situ curing (i.e. the polymer is cured at the location of its deposition), can be preferably achieved by exposing the polymer thin film to UV-wavelengths (during its deposition) or a plasma.

The fabrication process according to the invention may include further processing steps: For example it is proposed to deposit the electrode of the probe on a, preferably deformable and/or nanoscale, corrugation. This corrugation may be formed in the soft polymer thin film during the deposition.

In particular, the invention suggests that the polymer thin film can be deposited by molecular beam deposition, electro-spray-deposition or dip coating. In all cases, it is preferable if the polymer thin film is cured and/or annealed, in particular by an exposure to UV wavelengths, during (not after) its deposition.

These techniques allow an extremely accurate control of the film thickness and offer a high uniformity of the films. Both of these qualities are highly beneficial for achieving a high performance neural probe.

Prior to or during deposition of the electrode onto the polymer thin film, the polymer thin film may be treated by a plasma (or alternatively by an exposure to UV-wavelengths), preferably an oxygen-plasma. As a result, the surface corrugation, in particular in the form of nanoscale wrinkles or nanoscale ripples, can thus be formed in the polymer thin film prior or during deposition of the electrode.

For example by applying an oxygen plasma to the polymer thin film (which may be preferably a type of PDMS), an oxidized surface layer (featuring hydroxy-groups) may be formed in the polymer thin film, which has a different coefficient of thermal expansion than that of the underlying bulk polymer material. If such a polymer film is cooling down, it may form nanoscale wrinkles.

The formation of such wrinkles may be reduced or completely prevented by depositing a metal layer (offering a Young's modulus of typically several GPa) on top of the polymer thin film. However, there remains a compressive stress between the oxidized layer of the polymer film and the metal layer.

For the heterostructure consisting of the electrode and underlying soft thin film of the probe, a so called critical stress may be defined, which is a function of the elastic moduli of the electrode and soft thin film material, respectively. By varying the thickness of the thin film forming the electrode (preferably deposited on oxygen-plasma-treated PDMS films) of the probe, the intrinsic compressive stress in the electrode may be controlled, in particular below the critical stress. In such a case, in particular if the electrode is formed from a metal, the electrode cannot form wrinkles and therefore release the compressive stress; hence the electrode will show a flat top surface.

In conclusion, it is possible to conserve a compressive stress in the thin film forming the electrode and to fabricate either flat or wrinkled electrodes (depending on the amount of stress conserved in the layer) with a negligible increase in the stiffness of the overall (electrode/soft thin film-) heterostructure.

Accordingly, in the fabrication process according to the invention, the at least one electrode may be deposited, preferably on a nanoscale surface corrugation of the polymer thin film, in such a manner that a compressive stress in the at least one electrode is smaller than a critical stress of a heterostructure consisting of the at least one electrode and the underlying polymer (soft) thin film. This feature is achievable, for example, when sputtering the electrode and generating the stress thermally.

In the fabrication process according to the invention the at least one electrode may alternatively or additionally be deposited in such a manner that a network of connected and electrically conducting islands/clusters is formed on and/or within the soft thin film (embedded electrode). In particular, an areal portion of the conducting network can be near, preferably above, the percolation threshold. During elastic deformation, the electrode network can possibly be stretched below the percolation threshold, in which case, the network will not be conductive anymore. Hence, it is preferable, if the density of the islands of the network is above the percolation threshold.

Before the formation of the polymer thin film a micro- and/or nanoscale corrugation at the contact area of the carrier body can be formed. The corrugation (micro- and/or nanoscale structure) defines the alignment of the wrinkles of the soft thin film at the contact area. The height between the gauges and the ridges of the corrugation can be from 500 nm to 5 μm.

Before the formation of the polymer thin film and/or after the formation of a micro- and/or nanoscale corrugation at the contact area of the carrier body, a conductor is formed on the carrier body. The electrode may be electrically connected to the conductor. For electrically contacting the electrode the conductor can be directly applied on the carrier body, e.g. beneath the soft thin film and/or the electrode. The conductor may be formed as a conductive track (conductive thin film), which is applied on the carrier body by a sputter coating technique like high-power impulse magnetron sputtering (HIPIMS). The (coating) thickness of the conductor may be from 50 nm to 500 nm. The conductor may be covered by an encapsulation, e.g. an insulator, outside of the contact area. Within the contact area the conductor may be covered with the soft thin film and/or an embedded electrode.

In order to form an embedded electrode, the electrode can be formed by metal atoms or clusters embedded in the soft thin film. Thus, a flexible electrode (“soft electrode”) can be achieved. For example, gold and/or and/or platinum and/or titanium atoms or clusters may be deposited and embedded into the soft thin film. For electrically supply of the embedded electrode (in particular the metal atoms or clusters) has a solid/areal contact to the conductor, e.g. the conductive track, underneath. The metal atoms and clusters can be homogeneously distributed within the soft thin film. In case of the embedded electrode (soft electrode) no metal film is or needs to be formed on top of the soft thin film.

Preferred embodiments of the present invention shall now be described in more detail, although the invention is not limited to these embodiments: for those skilled in the art it is obvious that further embodiments of the invention may be obtained by combining features of one or more of the patent claims with each other and/or with one or more features of an embodiment described or illustrated herein.

### BRIEF DESCRIPTION OF THE DRAWINGS

With reference to the accompanying drawings, where features with corresponding technical function are referenced with same numerals even when these features differ in shape or design,

FIGS. 1A-1E illustrate a specific process for fabricating a neural probe according to the invention,

FIGS. 2A-2D illustrate another possible process for fabricating a neural probe according to the invention,

FIGS. 3A-3C illustrate yet another possible process for fabricating a neural probe according to the invention,

FIG. 4 illustrates neural probe bundle according to the invention, and finally

FIGS. 5A-5D provide detailed views of the distal end head of a neural probe according to the invention,

FIGS. 6&7 show the fabrication steps of a neural probe, wherein FIG. 7 shows the fabrication steps in Section A-A of the neural probe of FIG. 6,

FIG. 8 shows a neural probe, wherein Au and/or Pt atoms and/or clusters are embedded in a soft thin film (embedded electrode), which is formed as a SH-PDMS network (covalently bonded to thiol sites within PDMS network),

FIG. 9 provides a detailed view of detail A of FIG. 8,

FIGS. 10&11 show a double-sided neural probe,

FIG. 12 shows a first embodiment of a neural probe, having a soft thin film (e.g. an elastomer film) with a thickness of e.g. 50 nm to 500 nm, wherein the height of the microstructure beneath the soft thin film is equal or greater (e.g. 500 nm to 5 μm) and wherein micro- and/or nanostructures are formed as wrinkles which are aligned perpendicular to edges of microstructures,

FIG. 13 shows a second embodiment of a neural probe, having a soft thin film (e.g. an elastomer film) with a thickness of e.g. 500 nm to 5 μm, wherein the height of the microstructure beneath the soft thin film is equal (e.g. 500 nm to 5 μm) and wherein micro- and/or nanostructures are formed as wrinkles which are highly aligned,

FIG. 14 shows a second embodiment of a neural probe, having a soft thin film (e.g. an elastomer film) with a thickness of e.g. 5 μm to 20 μm, wherein the height of the microstructure beneath the soft thin film is smaller (e.g. 500 nm to 5 μm) and wherein micro- and/or nanostructures are formed as wrinkles which are highly aligned,

FIG. 15 shows a diagram showing the relation between strain and resistance of the neural probe, in particular the contact area of the neural probe.

### DETAILED DESCRIPTION

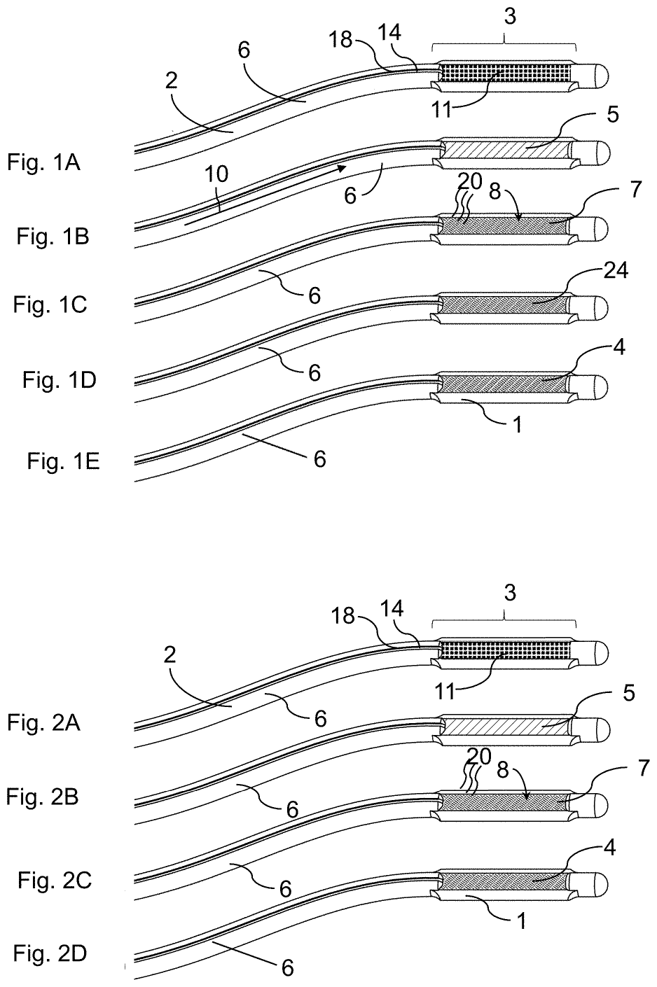

FIGS. 1A-1E illustrate a fabrication process for a neural probe 1, comprising five major process steps:

A polymer fiber 6 is used as the carrier body 2 of the neural probe 1, whose distal end is first micro-structured by thermal imprinting, as shown in FIG. 1A. As a result, the neural probe 1 features a contact area 3, which is formed as an approximately flat rectangular area with a microstructure 11 arranged within this area. In addition, a rounded distal end portion of the fiber 6 has been formed by the thermal imprinting step. As another result of the micro-structuring of the fiber 6, the surface area of the fiber part, which forms the contact area 3, has been greatly increased, such that a larger area can be used as a neural interface.

Next (FIG. 1B), an approximately 500 nm thick elastomeric layer of vinyl terminated PDMS (other types of functionalized siloxane based polymers may be used as well) is deposited by molecular beam deposition (MBD). This layer will later serve as an intermediate soft thin film 5, carrying an electrode 4 of the neural probe 1 on the stiffer material of the fiber 6, which serves as the carrier body 2 of the probe 1.

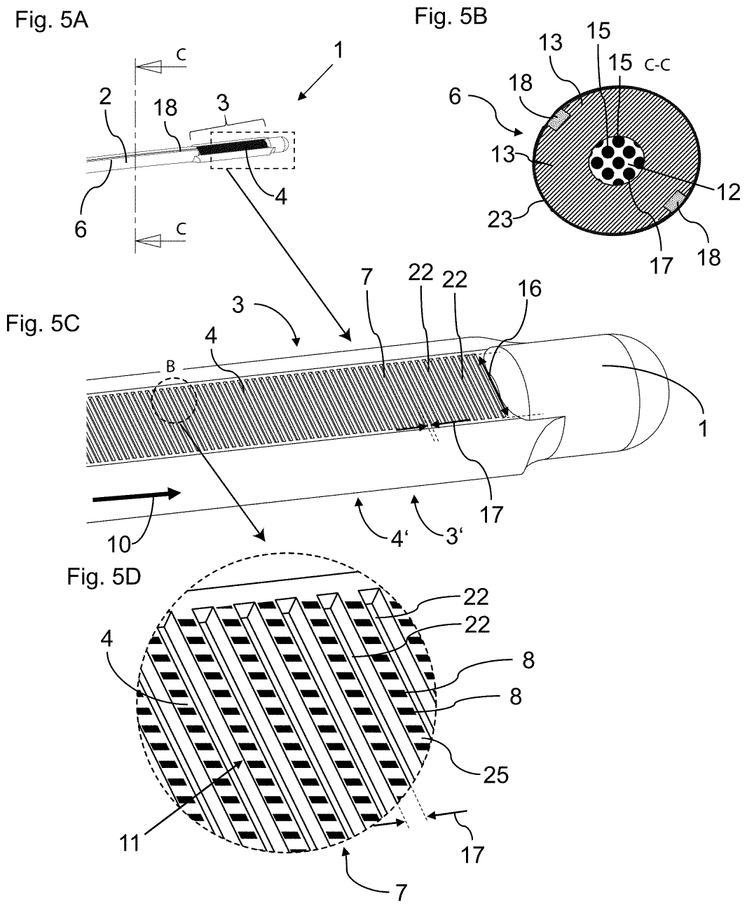

By treating the PDMS thin film 5 with an oxygen plasma 20 (FIG. 1c), as depicted in FIG. 1C, nanostructures are formed in the soft thin film 5; in particular, the nanostructures are formed on the microstructure 11 within the contact area 3. The nanostructures consist of a surface corrugation 7, that is made up by wrinkles and nanoscale ripples 8, which self-align to the microstructure 11, as will be explained in greater detail with respect to FIG. 5.

In a following process step (FIG. 1D), a second PDMS layer with a thickness of approximately 10 to 100 nm is deposited from the gas phase onto the plasma treated sub-micrometer (e.g. 500 nm) thick PDMS thin film 5. This second PDMS layer features thiol functionalized end groups and serves as an adhesion promotion layer 24 for the electrode 4, which is deposited in the following process step (FIG. 1E).

The first layer of vinyl-terminated PDMS is cured during deposition using ultraviolet (UV) light irradiation from an H2D2 deuterium light source (i.e. during the process step corresponding to FIG. 1B). The second layer of thiol-functionalized PDMS (serving as an adhesion promotion layer 24) is cured at another wavelength using a Hg—Xe UV light source (i.e. during the process step corresponding to FIG. 1e). In other words, both PDMS layers are cured during deposition by an UV exposure, respectively. This process can be referred to as in-situ curing, as the respective thin films are deposited from the gas phase (deposition from the liquid phase is also possible) and cured at the location, where they have been deposited, respectively.

Finally, as shown in FIG. 1E, a thin metal film of a thickness in the order of 7 to 30 nm is deposited within the contact area 3 of the neural probe 1 by thermal evaporation. The metal thin film forms an electrode 4, which can be used for stimulating nervous tissue. For this purpose, the fiber 6 features an electrical wiring 18 and an electrical supply line 14 which are electrically connected to the electrode 4. It is also possible to use standard patterning techniques (in particular shadow masks) to form several individual electrodes 4 within the contact area 3. In such a case, each electrode 4 may be contacted separately with the electrical wiring 18 such that the electrical potential of each electrode 4 can be measured or controlled.

FIGS. 2A-2D illustrates an alternative process for fabricating a neural probe 1 according to the invention. Again a polymer fiber 6 is micro-structured by thermal imprinting (FIG. 2A) to form a micro-structure 11 within a contact area 3, followed by electro-spray deposition of a 500 nm thick PDMS thin film 5 with vinyl terminated end groups. After curing the PDMS thin film 5 on the fiber 6, the distal tip of the fiber 6 is nanostructured by applying a plasma 20 to the PDMS thin film 5, such that a nanoscale surface corrugation 7 is formed which features nanoscale ripples 8.

In contrast to the process shown in FIGS. 1A-1E, no additional adhesion promotion layer 24 is used in the process illustrated by FIGS. 2A-2D. Rather, a metal electrode 4 in the form of a thin (7 to 30 nm thickness) gold film is applied directly onto the soft PDMS thin film 5, using a DC-sputtering technique. Due to this specific deposition technique, an electrode 4 is formed, which shows an intrinsic compression.

By choosing an appropriate thickness of the thin film forming the electrode, the compressive intrinsic stress can be held smaller or equal to the critical stress (see above). In this case, the increase in the stiffness in the overall heterostructure will be negligible. In conclusion, by conserving compressive stress in the electrode 4, using a suitable deposition technique such as sputtering, a high elasticity of the heterostructure can be achieved; in fact, this elasticity can be comparable to that of the soft thin film material (which may be PDMS, for example).

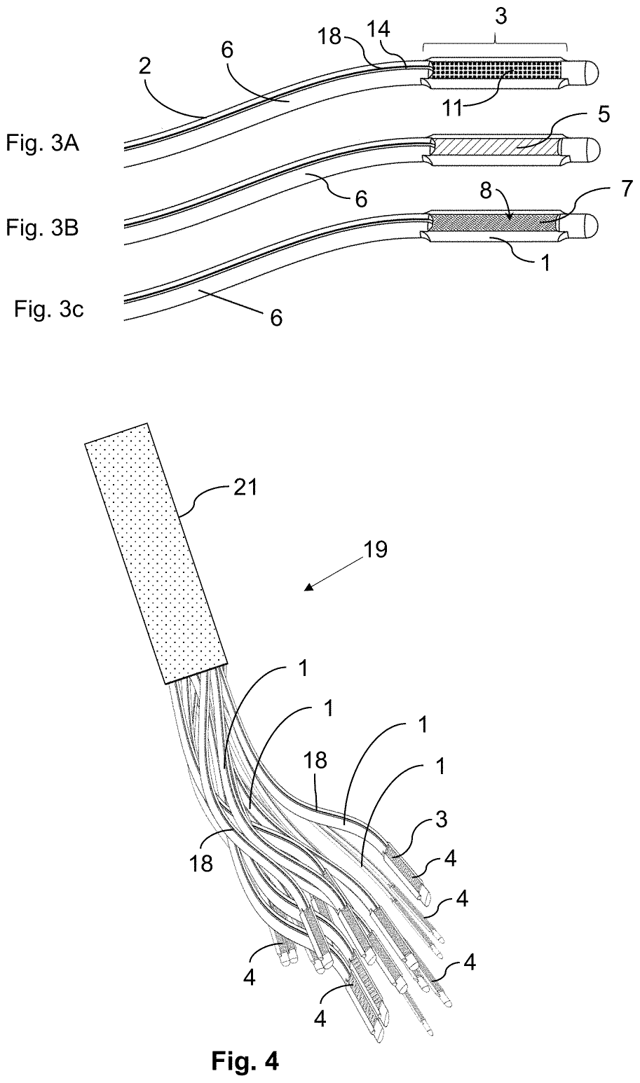

Yet another possible fabrication process for a neural probe 1 according to the invention with even fewer process steps is illustrated in FIGS. 3A-3C: After micro-structuring the fiber 6 by thermal imprinting (FIGS. 3a), a 50 to 500 nm thick soft thin film 5 of PDMS is deposited, but this time the PDMS features a thiol-end-group and is cross-linked using UV-radiation. Also different from the example of FIG. 2, the metal electrode 4 (consisting of gold) is deposited not by sputtering but by molecular beam epitaxy (thermal evaporation may be used as well). In this process, the nanoscale corrugation 7 is formed due to thermal stress during the deposition of the metal electrode 4 on the thiol-functionalized PDMS thin film 5; in fact, by this process the metal atoms are incorporated into the SH-PDMS matrix such that a highly compliant and soft electrode is formed.

FIG. 4 illustrates another aspect of the invention directed towards using a bundle 19 of neural probes 1, each designed according to the invention (i.e. in particular with several individual electrodes 4), as a powerful medical device for electrostimulation and/or -recording of nervous tissue by employing a high number of individual controllable electrodes 4.

As illustrated by FIG. 4, the bundle 19 may consist of 2/4/8/16/32/64/128/256/512 or even 1024 individual neural probes 1. For example when using probes with 2×2 individual electrodes 4 each, the total number of electrodes 4 of the bundle 19 may exceed 4096. This opens up completely new opportunities for accurately steering electrostimulation currents or high resolution electrical recording of nervous signals.

In particular when introducing the bundle 18 into brain tissue, it may be held together by a sleeve 21 (preferably from metal and for example with an outer diameter of less than 2 mm). After insertion, the sleeve 21 may be withdrawn.

In addition, each of the neural probes 1 of the bundle 19 may have an extractable core 12, for example formed by a single or several metal wires 15 (in other words, a neural probe 1 according to the invention may feature a hollow core 12, in particular into which a metal wire 15 or alike can be introduced and re-extracted). The wires 15 may be pre-stressed to predefine a certain shape of the individual neural probe 1. After inserting the neural probe bundle 19 into tissue, the sleeve 21 as well as the extractable cores 12, in particular the wires 15 detailed above, may be extracted or withdrawn. Consequently, the individual neural probes 1, which may have diameters as low as 50 μm, will spread and fan out, as depicted in FIG. 4. Thus, the bundle 19 as a whole may be rendered highly compliant and flexible, after inserting it into nervous tissue.

Moreover, by moving single neural probes 1 of the bundle 19 along their individual longitudinal axis 10 and/or by using neural probes 1 of differing lengths, the relative axial and or radial position or orientation of electrodes 4 of the individual neural probes 1 may be modified within the bundle 19. Using this approach, the location of the neural interfaces built up by each individual electrode 4 of the bundle 19 can be fine-tuned, such that the bundle 19 offers multiple electrodes 4, each positioned at different axial and/or radial positions along the bundle 19.

When using a probe bundle 19 according to the invention, some probes 1 of the bundle 19 may be used for stimulating neural tissue while other probes 1 of the bundle 19 may be used for recording neural signals from the tissue. For this purpose, some probes 1 of the bundle 19 may be configured for stimulating nervous tissue while the remaining probes 1 of the bundle 19 may be configured for recording neural signals.

Finally, FIGS. 5A-5D details microscale as well as nanoscale aspects of a neural probe 1 according to the invention. The distal end portion of the neural probe 1 shown in FIG. 5A) features a contact area 3 with an electrode 4 that may be fabricated as has been discussed for FIGS. 1-3.

FIG. 5B provides a detailed cross-sectional view of the neural probe 1 along the axis C-C shown in FIG. 5A. The fiber 6 forming the carrier body 2 of the probe 1 comprises a core 12 surrounded by a cladding 13. The core consists of several metal wires 15, which can be extracted, thus leaving a hollow core 12 behind. On the outer surface of the cladding 13, an electrical wiring 18 has been applied by printing. This wiring 18 is insulated from the exterior by an additional encapsulation 23, which surrounds the fiber 6.

As is visible from FIG. 5C and in particular from the detail inset depicted by FIG. 5D, a microstructure 11 has been formed by thermal imprinting in the contact area 3. The microscale structure 11 consists of numerous pit-shaped recesses 22 with a width below 1 μm. Each recess 22 is longer in a first direction 16 (corresponding to the length of the pit) than in a second direction 17 (corresponding to the width of the pit). Therefore, the microscale structure 11 features a dimension in the first direction 16 that is larger than a dimension in the second direction 17.

In between the single recesses 22, slim bridges 25 are formed by the micro-structure 11, which run across the full width of the contact area 3. Each bridge 25 shows a width of less than 500 nm. As a result, the ripples 8 of the nanoscale corrugation 7 in the soft thin film 5 (separating the electrode 4 from the fiber 6) forming on top of these bridges 25 are oriented approximately parallel to the direction with the shorter dimension, i.e. approximately parallel to the second direction 17 (c.f. FIG. 5d). This specific orientation of the nanoscale ripples 8 is a result of a self-alignment during formation of the corrugation 7 by plasma treatment (or alternatively by UV-irradiation).

In the example of FIGS. 5A-5D, the electrode 4 itself shows as surface corrugation 7 and therefore a largely increased surface area to be used as a neural interface to nervous tissue. This is a large benefit for the long-term stability of the interface.

Moreover, the nanoscale ripples 8 show an anisotropy of the elastic modulus on a submicrometer scale: it has been found that hills formed by the ripples 8 are approximately by a factor of 2 stiffer than the valleys formed between the hills and it is speculated that such a feature is beneficial for enhanced adhesion of cells forming the nervous tissue.

Indicated by FIG. 5D is also that, in fact, two contact areas 3 and 3′, each equipped with a stimulation and/or recording electrode 4/4′, are formed on two opposing sides of the fiber 6; each of the two contact areas 3 and 3′ is formed as has been described before with respect to FIGS. 5C and 5D. In summary, for improving the electroactivity and long-term stability of a neural interface, a novel neural probe 1 is proposed that is formed from a fiber 6, preferably by thermal imprinting, and wherein a polymer thin film 5 is employed for carrying a conducting thin film to be used as a recording or stimulation electrode 4. Due to this specific choice of materials and design, the electrode 4 is rendered compliant with respect to the fiber 6 on a nanometer to micrometer scale and offers a surface that is tailor-made for adhering to nervous tissue.

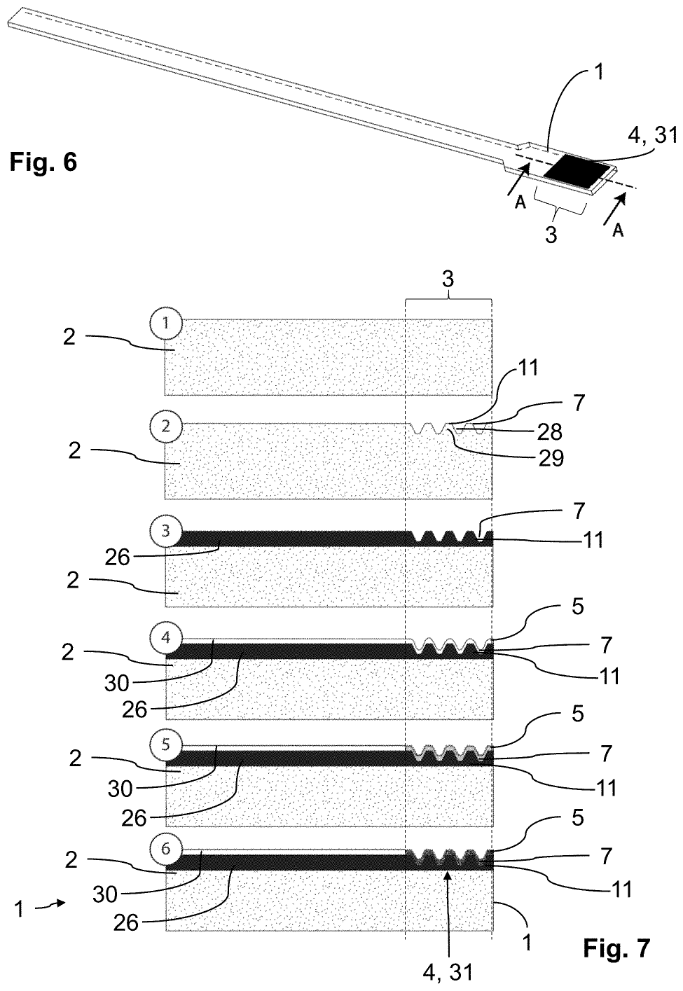

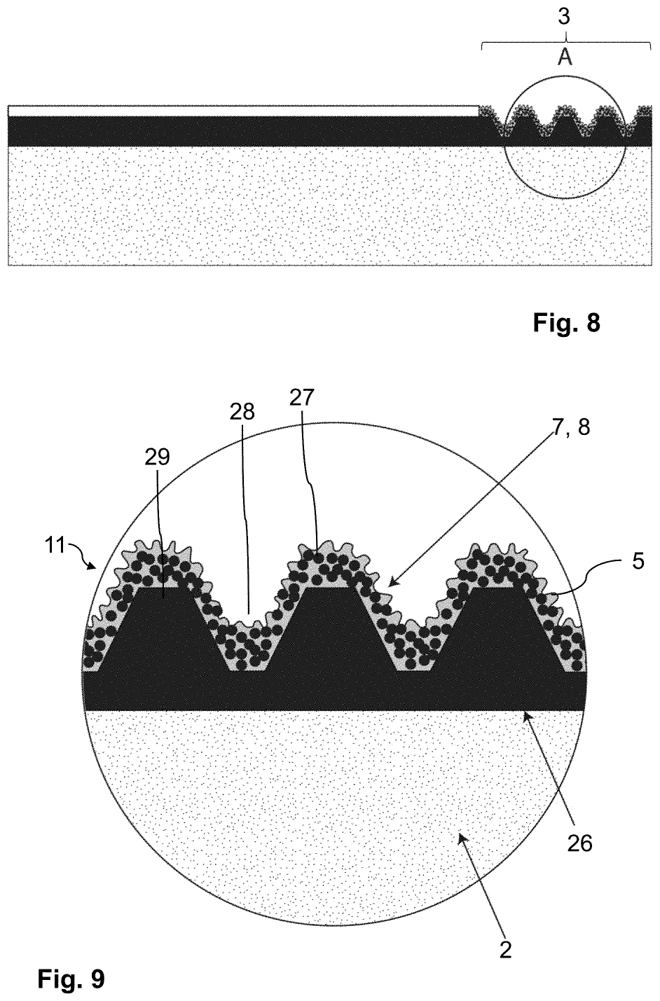

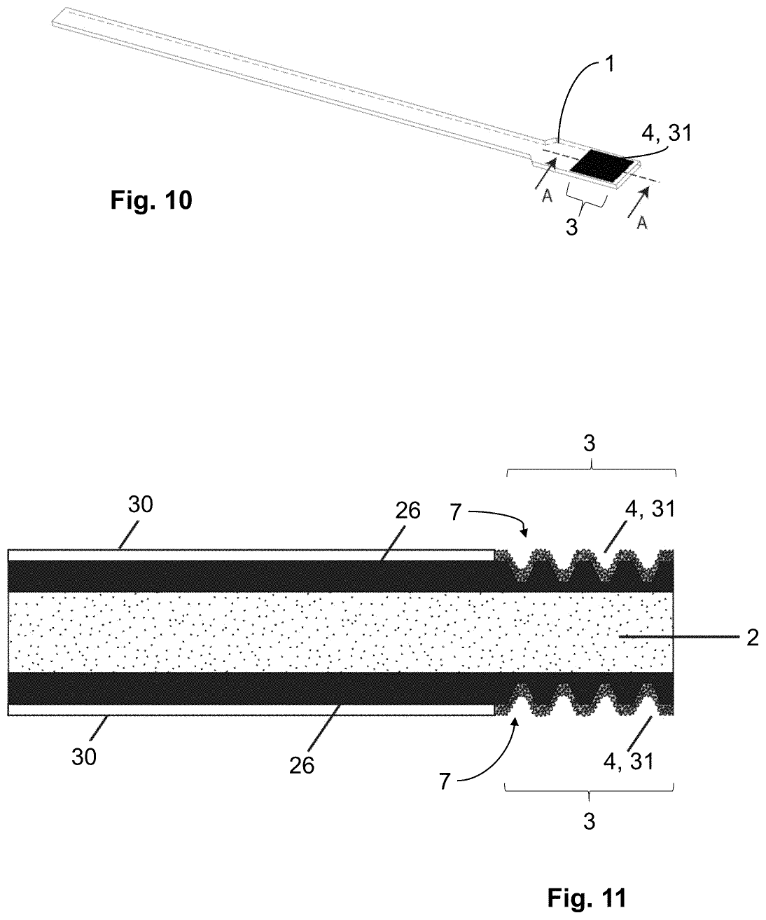

FIGS. 6 and 7 show another embodiment of a fabrication process which only differs from the fabrication process, which has been described above, by the steps 3 and 6.

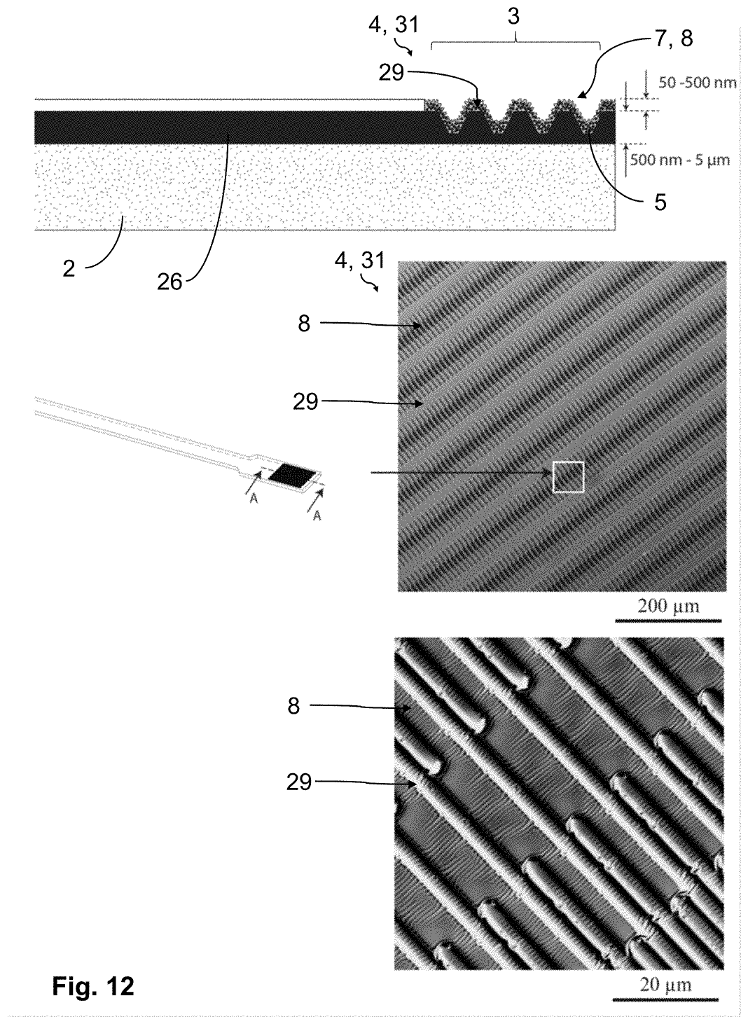

In a first step a micro- or nanoscale corrugation 7, in particular a microscale structure 11 (also called microstructure) with several ridges 29 and gauges 28 is formed at the contact area 3 of the carrier body 2. The ridges 29 and gauges 28 can be mostly aligned in parallel to each other. This step may be performed by thermal imprinting and/or hot embossing and/or UV-assisted imprinting or other molding techniques. The height of the ridges 29 to the bottom of the gauges 28 can be from 500 nm to 5 μm, as can be seen in FIGS. 12 to 14.

In the next step a conductor 26 is implanted on the carrier body 2. For example, the conductor 26 can be a thin film e.g. made of gold and/or platinum and/or titanium. It can be formed by high-power impulse magnetron sputtering (HiPIMS).

Then, a (soft) thin polymer film 5 is deposited on the corrugated contact area 3. The polymer film 5 may be an elastomer film, e.g. a silicone film, preferably polydimethylsiloxane (PDMS). The polymer film 5 may be thiol-functionalized for forming covalent bonds. The height of the polymer film 5 can be from 50 nm to 500 nm. Subsequently, the polymer film 5 may optionally be treated by plasma as described before, for enabling the formation of a nanoscale corrugation, e.g. nanoscale ripples/nanoscale wrinkles 8.

For forming an embedded electrode 31, metal atoms 27 are deposited into the polymer film 5. The embedded electrode 31 is electrically connected to the conductor 26 underneath the film 5.

FIGS. 12 to 14 show different embodiments of a nanoscale corrugation 7 (nanoscale wrinkles 8/nanoscale ripples 8) on the surface of the film 5 of the embedded electrode 31 in the contact area 3. The wrinkles 8 alignment depends on the thickness of the thin film 5. The surface of the embedded electrode 27 thus can be adjusted by altering the height of the film 5 on the carrier body 2. The FIGS. 12 to 14 also include pictures with high magnification of the embedded electrodes 31 recorded by scanning the electrodes 4, 31 with atomic force microscopy.

In FIG. 12 the polymer film 5 has a thickness from 50 nm to 500 nm. The gauges 28 of the corrugation 7 formed in the carrier body 2—which can be a microscale structure 11—are not completely filled up by the film 5. Thus, the corrugation 7/microscale structure 11 (also called microstructures) formed in the carrier body 2 also moulds the shape/alignment of the corrugation 7 formed in the film 5. The corrugation 7 formed in the film 5 may have the form of nanoscale wrinkles 8 or ripples 8. The peaks of the ridges 29 of the microscale structure 11 of the carrier body 2 are imprinted in the above polymer film 5, in particular the surface of the polymer film 5. Moreover, the alignment of the wrinkles 8 of the corrugation 7 depends on the thickness of the film 5 applied on the microstructure 11. The wrinkles 8 in FIG. 12 are mostly aligned in parallel to each other. Further, the wrinkles 8 are mostly aligned perpendicular to edges of the ridges 29 of the microscale structure 11 of the carrier body 2.

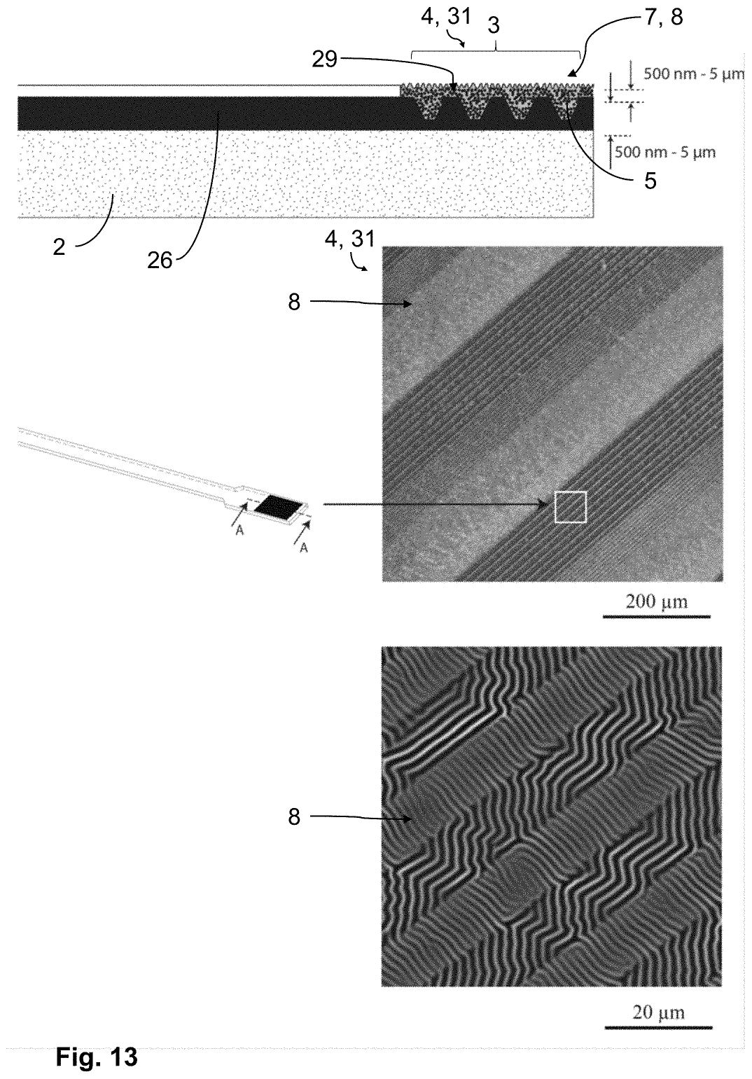

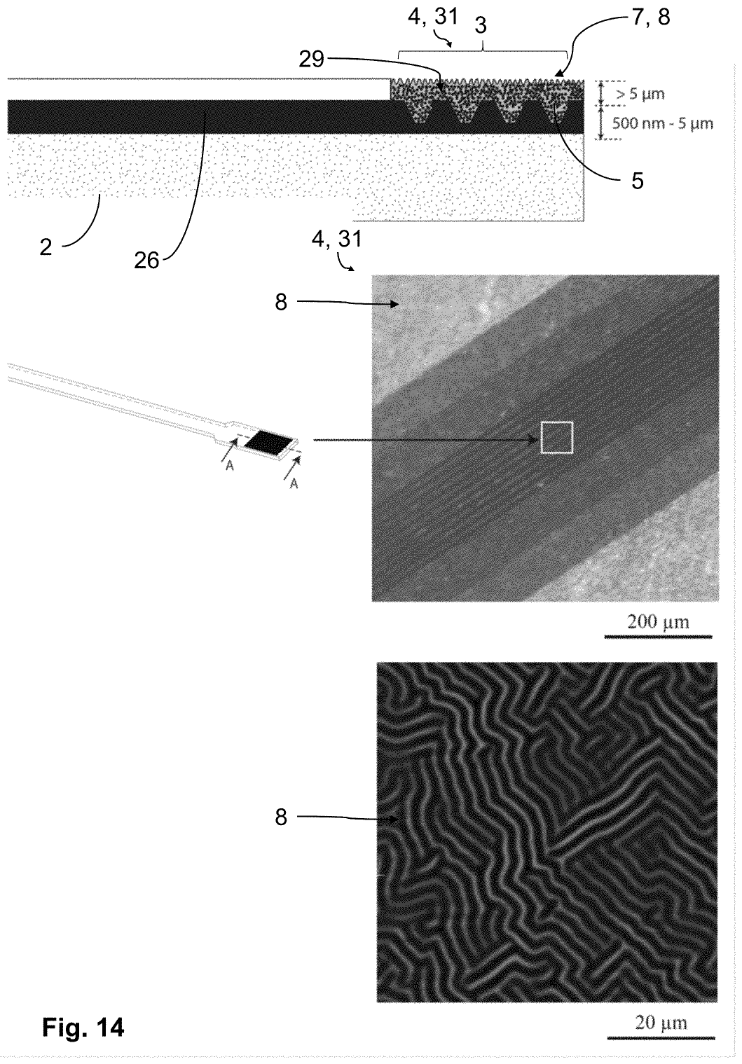

In contrast, FIGS. 13 and 14 show two further embodiments, wherein the microscale structures 11 formed in the carrier body 2 are completely covered by the film 5. In particular, the gauges 28 of the corrugation 7 formed in the carrier body 2 are completely filled out by the film 5. For example, the thickness of the film 5 can be from 500 nm to 20 μm, preferably from 500 nm to 5 μm (see FIG. 13) and/or 5 μm to 20 μm (see FIG. 14).

By using a high magnification, e.g. by using Atomic Force Microscopy, it is possible to recognize that the alignment of the wrinkles 8 in FIGS. 13 and 14 differs from the alignment of the wrinkles 8 in FIG. 12.

In the embodiment of FIG. 13 the wrinkles 8 are only partly aligned in parallel to each other. The other part of the surface shows a random alignment of the wrinkles 8. That means, the microstructure 11 underneath influences the alignment of the wrinkles 8 to a lesser extent. Here, the height of the wrinkles 8 amounts to about 10% of the thickness of the film 5.

In the embodiment of FIG. 14 the wrinkles 8 are mostly aligned randomly to each other and form serpentine nanoscale structures 8. That means, no influence of the microstructure 11 underneath the film 5 on the shaping and/or the alignment of the wrinkles 8 can be found.

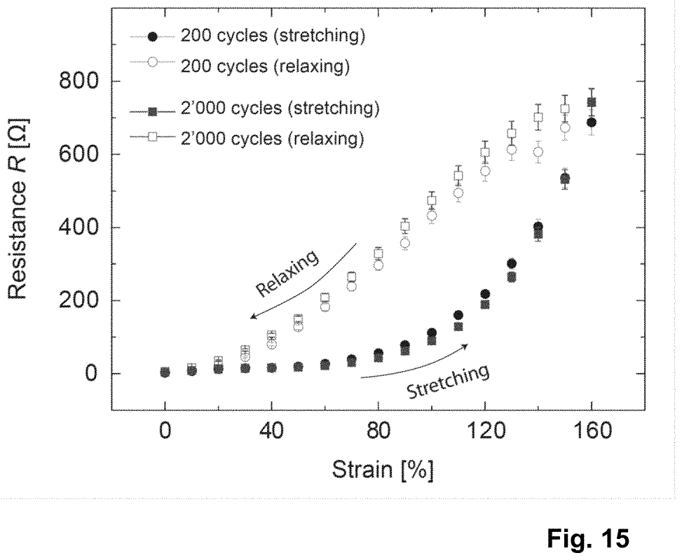

FIG. 15 shows a diagram showing the relation between strain and resistance of the neural probe 1, in particular the contact area of the neural probe (circles: 200 cycles/square: 2000 cycles). The elastic carrier body 2 of the neural probe 1 is stretchable. In contrast to known electrodes or metal films, the (flexible) electrode 4, 31 does not loose its conductivity when it is stretched. The carrier body 3 and/or the electrode 4, 31 is stretchable up to a strain of 160%, but at least more than 3%, preferably at least 5%. The embedded metal atoms 27 create a three-dimensional (3D) network within the film 5, which allows the stretching of the electrode 4, 31 without losing its conductivity. The film 5 with the embedded 3D network of metal atoms is connected to the carrier body 3. Usually, for probes carrier body materials are used, which do not show this quality that they can be stretched to the above-mentioned extent without tearing apart. Other carrier body materials usually tear apart at a strain of 3%, since they are not elastic, but stiff. The same applies for electrodes made of metal films, which are commonly used to form probes. In the event of tearing the resistance would become infinite and the conductivity is lost. The soft carrier body 3 of the stretchable neural probe 1 is compliant with the material of the polymer film 5 and allows therefore the creation of “stretchable electronics”.

### LIST OF REFERENCE NUMERALS

1 neural probe
2 carrier body
3 contact area
4 electrode
5 soft thin film
6 fiber
7 corrugation
8 nanoscale ripples; nanoscale wrinkles
9 sidewall (of 7)
10 longitudinal axis (of 6)
11 microscale structure
12 core (of 6)
13 cladding (of 6)
14 electrical supply line
15 metal wire
16 (dimension in) first direction
17 (dimension in) second direction
18 electrical wiring
19 bundle
20 plasma
21 sleeve
22 recess
23 encapsulation
24 adhesion promotion layer
25 bridge
26 conductor, in particular conductive track
27 embedded metal atoms or clusters
28 gauge of the micro- or nanoscale corrugation
29 ridge
30 encapsulation
31 embedded electrode

1. A neural probe (1) comprising:
a carrier body (2) forming a contact area (3),
at least one electrode (4) arranged within the contact area (3),
a soft thin film (5) carries the at least one electrode (4),
the soft thin film (5) is carried by a carrier body (2), and the soft thin film (5) is softer than the carrier body (2).

2. The neural probe (1) as claimed in claim 1, wherein the at least one electrode (4) is at least one of an electrical stimulation or an electrical recording electrode, and wherein the carrier body (3) is a fiber (6) with an outer diameter that is at least one of less than 0.8 mm or more than 0.05 mm.

3. The neural probe (1) as claimed in claim 1, wherein the at least one electrode (4) is formed as a thin film electrode (4) with a thickness of less than 50 nm or the at least one electrode (4) at least one of forms or follows a micro- or nanoscale corrugation (7).

4. The neural probe (1) as claimed in claim 3, wherein the corrugation (7) is a corrugation (7) of an outer surface of the electrode (4) that is adapted to be brought into contact with brain tissue and at least one of
wherein the corrugation (7) shows nanoscale ripples (8) or the soft thin film (5) itself shows a nanoscale corrugation (7) which is covered by the at least one electrode (4).

5. The neural probe (1) as claimed in claim 1, wherein the contact area (3) forms a microscale structure (11) including t least one of a microscale protrusion or a microscale recess (22), and at least one of the at least one electrode (4) or the microscale structure (11) is covered by the soft thin film (5).

6. The neural probe (1) as claimed in claim 1, wherein the contact area (3) is formed by at least one of thermal assisted printing, UV-assisted imprinting, or from the fiber material.

7. The neural probe (1) as claimed in claim 2, wherein at least one of: the fiber (6) is a polymer fiber, the fiber (6) has a core (12) which is electrically conducting, the fiber (6) features an electrical wiring (18), or the fiber and/or has a core (12) which is stiffer than a surrounding cladding (13) of the fiber (6).

8. The neural probe (1) as claimed in claim 2, wherein at least one of a Young's Modulus of the polymer of the soft thin film (5) is at least a factor of 10³ lower than a Young's Modulus of a polymer of the fiber (6) or—the polymer of the soft thin film (5) is an elastomer.

9. The neural probe (1) as claimed in claim 1, wherein the soft thin film (5) has a thickness of less than 10 μm, the at least one electrode (4) is linked to the soft thin film (5) by an intermediate adhesion promotion layer formed of thiol-functionalized PDMS.

10. The neural probe (1) as claimed in claim 1, wherein an electrical wiring (18) is arranged on an outer surface of the fiber (6), the fiber (6) features at least one of electrical contacts pads on the outer surface connected to the electrical wiring (18) or an encapsulation (23) which insulates the wiring (18).

11. The neural probe (1) as claimed in claim 1, wherein the carrier body (2) carries a conductor (26).

12. The neural probe (1) of claim 11, wherein the conductor (26) is produced by physical vapor deposition by high-power impulse magnetron sputtering.

13. The neural probe (1) as claimed in claim 1, wherein the electrode (4) is formed by metal atoms or clusters (27), embedded in the soft thin film (5)

14.-18. (canceled)

19. A method for fabricating a neural probe (1) including polymer thin film (5) which carries at least one electrode (4) of the probe (1), and the carrier body (2) forms a contact area (3) within which the at least one electrode (4) is arranged, the method comprising depositing the polymer thin film (5) from a gas or liquid phase and that is cross-linked during deposition by exposure to UV-wavelengths or a plasma.

20.-21. (canceled)

22. The method according to claim 19, wherein at least one of: the at least one electrode (4) is deposited such that a network of connected and electrically conducting islands is formed on the soft thin film (5), or before the formation of the polymer thin film (5) at least one of a micro- or nanoscale corrugation (7) at the contact area (3) of the carrier body (2) is formed.

23.-25. (canceled)
